(19) United States
(12) Patent Application Publication
(10) Pub. No.: US 2025/0214919 A1
(43) Pub. Date: Jul. 3, 2025

(54) THIN FILM MODIFICATION COMPOSITION, METHOD OF FORMING THIN FILM USING THIN FILM MODIFICATION COMPOSITION, SEMICONDUCTOR SUBSTRATE INCLUDING THIN FILM, AND SEMICONDUCTOR DEVICE INCLUDING SEMICONDUCTOR

(71) Applicant: SOULBRAIN CO., LTD., Gyeonggi-do (KR)

(72) Inventors: Seung Hyun LEE, Gyeonggi-do (KR); Jae Sun JUNG, Gyeonggi-do (KR); Deok Hyun KIM, Gyeonggi-do (KR)

(21) Appl. No.: 18/847,412

(22) PCT Filed: Mar. 17, 2023
(86) PCT No.: PCT/KR2023/003586

§ 371 Date: Sep. 16, 2024

(30) Foreign Application Priority Data
Apr. 5, 2022 (KR) 10-2022-0042350
Oct. 25, 2022 (KR) 10-2022-0138090

(51) Int. Cl. C07C 43/11; C23C 16/04; C23C 16/455; H01L 21/02

(52) U.S. Cl. CPC C07C 43/11; C23C 16/04; C23C 16/45525; H01L 21/02274; H01L 21/0228

(57) ABSTRACT

The present invention relates to a thin film modification composition, a method of forming a thin film using the thin film modification composition, a semiconductor substrate including the thin film, and a semiconductor device including the semiconductor substrate. According to the present invention, by using a step coverage improver and a diffusion improver in combination, the reaction speed may be improved and the thin film growth rate may be appropriately reduced. Thus, even when forming a thin film on a substrate with a complex structure under high-temperature conditions, step coverage and the thickness uniformity of a thin film may be greatly improved, impurities may be reduced, and film quality may be improved.

### TECHNICAL FIELD

The present invention relates to a thin film modification composition, a method of forming a thin film using the thin film modification composition, a semiconductor substrate including the thin film, and a semiconductor device including the semiconductor substrate. More particularly, the present invention relates to a thin film modification composition consisting of a step coverage improver and a diffusion improver, a method of forming a thin film using the thin film modification composition, and a semiconductor substrate including the thin film. According to the present invention, by forming a deposition layer with a uniform thickness as a shielded area on a substrate due to the difference in adsorption distribution of the compound, the deposition rate of the thin film may be reduced, and the thin film growth rate may be appropriately reduced. In addition, even when forming a thin film on a substrate with a complex structure, step coverage and the thickness uniformity of a thin film may be improved, and impurities may be greatly reduced.

### BACKGROUND ART

As the integration of memory and non-memory semiconductor devices increases, the microstructure of a substrate is becoming increasingly complex.

For example, the ratio of the width to depth of the microstructure (hereinafter referred to as the ‘aspect ratio’) is increasing to 20:1 or more, and even to 100:1 or more. As the aspect ratio increases, it becomes difficult to form a deposition layer with a uniform thickness along the plane of the complex microstructure.

Accordingly, step coverage, which defines the thickness ratio of deposition layers formed at the top and bottom in the depth direction of the microstructure, remains at the level of 90%. Accordingly, the expression of the electrical characteristics of a device becomes increasingly difficult. Since a step coverage of 100% means that deposition layers formed on the upper and lower parts of the microstructure have the same thickness, it is necessary to develop a technology that can achieve step coverage close to 100%.

That is, to provide excellent and uniform physical properties to a thin film deposited on a substrate, it is essential that the thin film has high step coverage. Accordingly, the atomic layer deposition (ALD) process, which uses surface reactions, is used rather than the chemical vapor deposition (CVD) process, which mainly uses gaseous reactions. However, there are still problems in implementing 100% step coverage.

When increasing deposition temperature to achieve 100% step coverage, it is difficult to achieve step coverage. First, in a deposition process using a precursor and a reactant, an increase in the deposition temperature leads to a steep increase in the thin film growth rate (GPC). In addition, even when the ALD process is performed at 300° C. to alleviate the increase in GPC due to the increase in deposition temperature, the deposition temperature increases during the process. Accordingly, the problem still remains.

In addition, high-temperature processes are required to realize metal oxide films with excellent film quality in semiconductor devices. A study has been reported in which the concentration of carbon and hydrogen remaining in a thin film was reduced by increasing the atomic layer deposition temperature to 400° C. (See the paper J. Vac. Sci. Technol. A, 35(2017) 01B130).

However, as the deposition temperature increases, it becomes difficult to ensure step coverage. First, in a deposition process using a precursor and a reactant, an increase in deposition temperature may lead to a sharp increase in thin film growth rate (GPC). In addition, even when a known shielding agent is applied to reduce the increase in GPC with increasing deposition temperature, it is confirmed that GPC increases by about 10% at 300° C. That is, when deposition is performed at temperatures above 360° C., it is difficult to expect the GPC reduction effect provided by the conventionally known shielding agent.

Therefore, there is a need to develop a thin film formation method that allows the formation of a thin film with a complex structure effectively even at high temperatures, reduces the residual amount of impurities, and improves step coverage and the thickness uniformity of a thin film and a semiconductor substrate including the thin film.

### DISCLOSURE

### Technical Problem

Therefore, the present invention has been made in view of the above problems, and it is one object of the present invention to provide a thin film modification composition, a method of forming a thin film using the thin film modification composition, and a semiconductor substrate including the thin film. According to the present invention, by using a step coverage improver and a diffusion improver in combination, a substrate may be effectively shielded, reaction speed may be improved, and thin film growth rate may be appropriately reduced. Thus, even when forming a thin film on a substrate with a complex structure, step coverage and the thickness uniformity of a thin film may be greatly improved, impurities may be reduced, and film quality may be improved.

It is another object of the present invention to improve the density and dielectric properties of a thin film by improving the crystallinity and oxidation fraction of the thin film.

The above and other objects can be accomplished by the present invention described below.

### Technical Solution

In accordance with one aspect of the present invention, provided is a thin film modification composition including 50 to 99 parts by weight of a step coverage improver including a compound represented by Chemical Formula 1 below and 1 to 50 parts by weight of a diffusion improver.

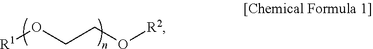

In Chemical Formula 1, R1 and R2 are independently H or an alkyl group having 1 to 5 carbon atoms, and n is an integer from 1 to 4.

The step coverage improver may have two or more oxygen (O), phosphorus (P), or sulfur (S) elements and may include a linear or cyclic saturated or unsaturated hydrocarbon having 3 to 15 carbon atoms.

The step coverage improver may include a compound having a structure containing oxygen (O), phosphorus (P), or sulfur (S) at each terminal of a central carbon atom connected to oxygen by a double bond.

The step coverage improver may include a compound having a structure containing oxygen (O), phosphorus (P), or sulfur (S) at one end of a central carbon atom connected to oxygen by a double bond and carbon (C) at the other end.

The step coverage improver may include one or more selected from compounds represented by Chemical Formulas 1-1 to 1-7 below.

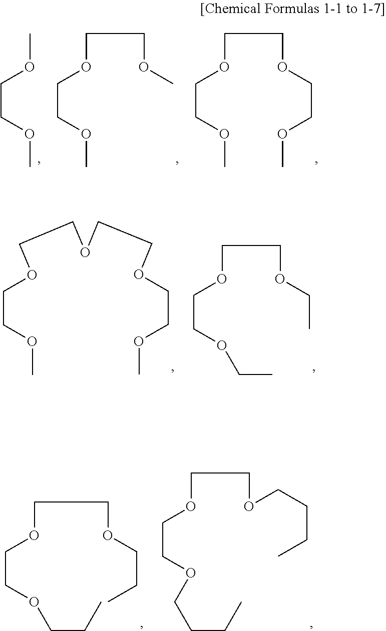

The step coverage improver may have a refractive index (measured at 20 to 25° C.) of 1.30 or more, 1.3 to 1.5, 1.35 to 1.48, or 1.36 to 1.46.

The diffusion improver may have a boiling point of 5 to 200° C., as a specific example, 50 to 150° C., preferably 100 to 140° C.

To improve the diffusion and uniformity of the above-described step coverage improver, the diffusion improver is preferably a liquid at room temperature.

The diffusion improver may include one or more selected from octane, diethyl ether, methylene chloride, toluene, hexane, and ethanol.

The diffusion improver may have a refractive index of 1.30 or more, 1.30 to 1.70, 1.35 to 1.60, or 1.36 to 1.50.

The thin film modification composition may have a deposition rate reduction rate of 5% or more as calculated by Equation 1 below.

$$Deposition rate reduction rate = [ \{ ( {DR}_{i} ) - ( D {R}_{f} ) \} / ( {DR}_{i} ) ] \times 100 \qquad [ Equation 1 ]$$

In Equation 1, deposition rate (DR, Å/cycle) is the speed at which a thin film is deposited. In the deposition of a thin film formed from a precursor and a reactant, DRᵢ (initial deposition rate) is the deposition rate of the thin film formed without adding a diffusion improver. DR_{f} (final deposition rate) is the deposition rate of the thin film formed by adding the diffusion improver during the above process. Here, the deposition rate (DR) is a value measured at room temperature and pressure using an ellipsometer for a thin film with a thickness of 3 to 30 nm, and is expressed in a unit of Å/cycle.

The thin film may control a reaction surface of a thin film formed from one or more precursor compounds selected from the group consisting of Al, Si, Ti, V, Co, Ni, Cu, Zn, Ga, Ge, Se, Zr, Nb, Mo, Ru, Rh, In, Sn, Sb, Te, Hf, Ta, W, Re, Os, Ir, La, Ce, and Nd.

The thin film may be used as a diffusion barrier film, an etching stop film, an electrode film, a dielectric film, a gate insulating film, a block oxide film, or a charge trap, and may control a reaction surface during formation thereof.

In accordance with another aspect of the present invention, provided is a method of forming a thin film, the method including:
injecting a thin film modification composition including 50 to 99 parts by weight of a step coverage improver including a compound represented by Chemical Formula 1 below and 1 to 50 parts by weight of a diffusion improver into a chamber to shield a surface of a loaded substrate.

The precursor compound used in the method of forming a thin film may be a compound represented by Chemical Formula 2 below.

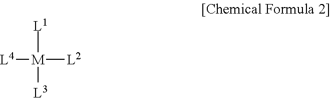

In Chemical Formula 2, M includes one or more selected from Al, Si, Ti, V, Co, Ni, Cu, Zn, Ga, Ge, Se, Zr, Nb, Mo, Ru, Rh, In, Sn, Sb, Te, Hf, Ta, W, Re, Os, Ir, La, Ce, and Nd; and L1, L2, L3, and L4 are —H, —X, —R, —OR, —NR, or Cp (cyclopentadiene) and may be the same or different. Here, —X may be F, Cl, Br, or I; —R may be linear or circular C1-C10 alkyl, C1-C10 alkene, or C1-C10 alkane; and L1, L2, L3, and L4 may be formed from 2 to 6 depending on the oxidation number of a central metal.

For example, when the central metal is divalent, L1 and L2 may be attached to the central metal as ligands. When the central metal is hexavalent, L1, L2, L3, L4, L5, and L6 may be attached to the central metal. The ligands corresponding to L1 to L6 may be the same or different.

In Chemical Formula 2, L1, L2, L3, and L4 may be —H, —X, or —R, and may be the same or different. Here, —R may be linear or circular C1-C10 alkyl, C1-C10 alkene, or C1-C10 alkane.

In Chemical Formula 2, L1, L2, L3, and L4 may be —H, —OR, —NR, or Cp (cyclopentadiene), and may be the same or different. Here, —R may be H, C1-C10 alkyl, C1-C10 alkene, C1-C10 alkane, iPr, or TBu.

In Chemical Formula 2, L1, L2, L3, and L4 may be —H or —X, and may be the same or different. Here, —X may be F, Cl, Br, or I.

In accordance with still another aspect of the present invention, provided is a method of forming a thin film, the method including:
i) vaporizing the above-described thin film modification composition to shield a surface of a substrate loaded in a chamber;
ii) performing 1st purging inside the chamber with a purge gas;
iii) vaporizing a precursor compound and adsorbing the precursor compound onto an area outside the shielded area;
iv) performing 2nd purging inside the chamber with a purge gas;
v) supplying a reaction gas inside the chamber; and
vi) performing 3rd purging inside the chamber with a purge gas.

The precursor compound may be a molecule composed of one or more selected from the group consisting of Al, Si, Ti, V, Co, Ni, Cu, Zn, Ga, Ge, Se, Zr, Nb, Mo, Ru, Rh, In, Sn, Sb, Te, Hf, Ta, W, Re, Os, Ir, La, Ce, and Nd, and may be a precursor having a vapor pressure of greater than 0.01 mTorr and 100 Torr or less at 25° C.

The chamber may be an ALD chamber, a CVD chamber, a PEALD chamber, or a PECVD chamber.

The thin film modification composition or the precursor compound may be vaporized, injected, and then subjected to plasma post-treatment.

In steps i) and iv), an amount of the purge gas injected into the chamber at each step may be 10 to 100,000 times the volume of the thin film modification composition injected.

The reaction gas may be an oxidizing agent, nitriding agent, or reducing agent, and the reaction gas, thin film modification composition, and precursor compound may be transferred into the chamber by a VFC, DLI, or LDS method.

The thin film may be a silicon nitride film, a silicon oxide film, a titanium nitride film, a titanium oxide film, a tungsten nitride film, a molybdenum nitride film, a hafnium oxide film, a zirconium oxide film, a tungsten oxide film, or an aluminum oxide film.

The substrate loaded into the chamber may be heated to 100 to 800° C., and the ratio of amount (mg/cycle) of the thin film modification composition and the precursor compound fed into the chamber may be 1:1 to 1:20.

In accordance with still another aspect of the present invention, provided is a semiconductor substrate including the thin film manufactured by the above-described method of forming a thin film.

The thin film may have a multilayer structure of two or more layers.

In accordance with yet another aspect of the present invention, provided is a semiconductor device including the above-described semiconductor substrate.

The semiconductor substrate may be low resistive metal gate interconnects, a high aspect ratio 3D metal-insulator-metal capacitor, a DRAM trench capacitor, 3D Gate-All-Around (GAA), or a 3D NAND flash memory.

### Advantageous Effects

According to the present invention, the present invention has an effect of providing a thin film modification composition that improves step coverage even when forming a thin film on a substrate with a complex structure under high-temperature conditions by effectively shielding adsorption on the surface of the substrate to improve reaction speed and reduce a thin film growth rate appropriately.

In addition, by effectively reducing process by-products during thin film formation, corrosion and deterioration can be prevented, and the film quality can be improved to improve the crystallinity of a thin film, thereby improving the electrical properties of the thin film.

In addition, when forming a thin film, process by-products can be reduced, and step coverage and thin film density can be improved. In addition, a method of forming a thin film and a semiconductor substrate fabricated by the method can be provided.

### DESCRIPTION OF DRAWINGS

FIG. 1 is a schematic diagram showing a deposition process sequence according to the present invention, focusing on one cycle.

FIG. 2 is a drawing comparing the calculated non-uniformity for each thin film obtained in Examples 1 and 2 and Comparative Examples 2 and 3 described below.

FIG. 3 is a TEM image measuring the step coverage at each of the upper and lower parts of a thin film obtained in Example 1 described below.

### BEST MODE

Hereinafter, a thin film modification composition of the present invention, a method of forming a thin film using the thin film modification composition, and a semiconductor substrate including the thin film will be described in detail.

In the present disclosure, unless otherwise specified, the term “step coverage improver” means improving step coverage by reducing, inhibiting, or blocking the adsorption of a precursor compound for forming a thin film onto a substrate and the adsorption of process by-products onto the substrate.

In the present disclosure, unless otherwise specified, the term “shielding” means reducing, inhibiting, or blocking adsorption of precursor compounds for forming a thin film onto a substrate, and also reducing, inhibiting, or blocking adsorption of process by-products onto the substrate.

In the present disclosure, unless otherwise specified, the term “improving diffusion” refers to improving uniformity by promoting the diffusion of a step coverage improver.

The present inventors confirmed that, by using a step coverage improver capable of shielding the adsorption of a precursor compound supplied to form a thin film on the surface of a substrate loaded inside a chamber and a diffusion improver capable of improving the uniformity of the generated thin film in combination, reaction speed was improved under high-temperature conditions. In addition, even when applied to a substrate with a complex structure, film quality was improved, the uniformity of a thin film was ensured, step coverage was greatly improved. In particular, thin-thickness deposition was possible, and the remaining O, Si, metal, and metal oxides as process by-products and carbon residues, which were difficult to reduce in the past, were reduced. Based on these results, the present inventors conducted further studies on a thin film modification composition to complete the present invention.

For example, the thin film may be provided with one or more precursors selected from the group consisting of Al, Si, Ti, V, Co, Ni, Cu, Zn, Ga, Ge, Se, Zr, Nb, Mo, Ru, Rh, In, Sn, Sb, Te, Hf, Ta, W, Re, Os, Ir, La, Ce, and Nd, and may provide an oxide film, a nitride film, or a metal film. In this case, the effects desired in the present invention may be sufficiently achieved.

As a specific example, the thin film may have a film composition of a silicon nitride film, a silicon oxide film, a titanium nitride film, a titanium oxide film, a tungsten nitride film, a molybdenum nitride film, a hafnium oxide film, a zirconium oxide film, a tungsten oxide film, or an aluminum oxide film.

The thin film means the above-described film composition also including SiH and SiOH.

In addition to a commonly used diffusion barrier film, the thin film may be used in semiconductor devices as an etching stop film, an electrode film, a dielectric film, a gate insulating film, a block oxide film, or a charge trap.

In the present invention, the precursor compound used in the formation of the thin film may be a molecule including one or more selected from the group consisting of Al, Si, Ti, V, Co, Ni, Cu, Zn, Ga, Ge, Se, Zr, Nb, Mo, Ru, Rh, In, Sn, Sb, Te, Hf, Ta, W, Re, Os, Ir, La, Ce, and Nd as central metal atoms (M) and one or more ligands selected from C, N, O, H, X (halogen), and Cp (cyclopentadiene), and may be a precursor having a vapor pressure of 1 mTorr to 100 Torr at 25° C.

For example, a compound represented by Chemical Formula 2 below may be used as the precursor compound.

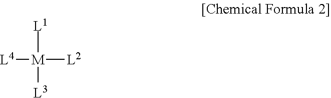

In Chemical Formula 2, M includes one or more selected from Al, Si, Ti, V, Co, Ni, Cu, Zn, Ga, Ge, Se, Zr, Nb, Mo, Ru, Rh, In, Sn, Sb, Te, Hf, Ta, W, Re, Os, Ir, La, Ce, and Nd; and L1, L2, L3, and L4 are —H, —X, —R, —OR, —NR, or Cp (cyclopentadiene) and are the same or different. Here, —X may be F, Cl, Br, or I; —R may be linear or circular C1-C10 alkyl, C1-C10 alkene, or C1-C10 alkane; and L1, L2, L3, and L4 may be formed from 2 to 6 depending on the oxidation number of a central metal.

For example, when the central metal is divalent, L1 and L2 may be attached to the central metal as ligands. When the central metal is hexavalent, L1, L2, L3, L4, L5, and L6 may be attached to the central metal. The ligands corresponding to L1 to L6 may be the same or different.

M may be a trivalent metal, tetravalent metal, pentavalent metal, or hexavalent metal, preferably hafnium (Hf), zirconium (Zr), aluminum (Al), niobium (Nb) or tellurium (Ta). In this case, the effect of reducing process by-products and the effect of improving thin film density may be increased, and step coverage and the electrical properties, insulating properties, and dielectric properties of the thin film may be excellent.

L1, L2, L3, and L4 are —H, —X, or —R, and may be the same or different. Here, —R may be C1-C10 alkyl, C1-C10 alkene, or C1-C10 alkane and may have a linear or cyclic structure.

In addition, L1, L2, L3, and L4 are —H, —OR, —NR, or Cp (cyclopentadiene), and may be the same or different. Here, —R may be H, C1-C10 alkyl, C1-C10 alkene, C1-C10 alkane, iPr, or tBu.

In addition, in Chemical Formula 2, L1, L2, L3, and L4 may be —H or —X, and may be the same or different. Here, —X may be F, Cl, Br, or I.

Specifically, examples of the silicon precursor compound may include one or more selected from SiH₄, SiHCl₃, SiH₂Cl₂, SiCl₄, Si₂Cl₆ Si₃Cl₈, Si₄Cl₁₀, SiH₂[NH(C₄H₉)₂], Si₂(NHC₂H₅)₄, Si₃NH₄ (CH₃)₃, SiH₃[N(CH₃)₂], SiH₂[N(CH₃)₂]₂, SiH[N(CH₃)₂]₃, and Si[N(CH₃)₂]₄.

In addition, examples of the hafnium precursor compound may include tris(dimethylamido)cyclopentadienyl hafnium of CpHf(NMe₂)₃ and (methyl-3-cyclopentadienylpropylamino)bis(dimethylamino)hafnium of Cp(CH₂)₃NM₃Hf(NMe₂)₂.

In addition, examples of the aluminum precursor compound may include trimethyl aluminum (TMA), tris(dimethylamido)aluminum (TDMAA), and aluminum chloride (AlCl₃).

The thin film modification composition may be composed of a step coverage improver and a diffusion improver.

Here, the step coverage improver may be a compound having at least one unshared electron pair selected from oxygen (O), sulfur (S), and phosphorus (P), preferably a compound having two or more oxygen (O), phosphorus (P), or sulfur (S) elements and including a linear or cyclic saturated or unsaturated hydrocarbon having 3 to 15 carbon atoms. In this case, by forming a shielded area that does not remain in a thin film during thin film formation, a relatively coarse thin film may be formed and side reactions may be suppressed. In addition, by controlling the thin film growth rate, process by-products within the thin film may be reduced, thereby reducing corrosion and deterioration. In addition, the crystallinity of the thin film may be improved, and a stoichiometric oxidation state may be reached when a metal oxide film is formed. In addition, even when a thin film is formed on a substrate with a complex structure under high-temperature conditions, step coverage and the thickness uniformity of a thin film may be greatly improved.

As a specific example, the step coverage improver includes a compound having a structure containing oxygen (O), phosphorus (P), or sulfur (S) at each terminal of a central carbon atom connected to oxygen by a double bond. Thus, the effect of reducing process by-products and the effect of improving thin film density may be increased, and step coverage and the electrical properties of the thin film may be excellent.

As a specific example, the step coverage improver may include one or more selected from compounds represented by Chemical Formula 1 below. In this case, by forming a shielded area that does not remain in a thin film during thin film formation, a relatively coarse thin film may be formed and side reactions may be suppressed. In addition, by controlling the thin film growth rate, process by-products within the thin film may be reduced, thereby reducing corrosion and deterioration. In addition, the crystallinity of a thin film may be improved. In addition, even when a thin film is formed on a substrate having a complex structure, step coverage and the thickness uniformity of a thin film may be greatly improved.

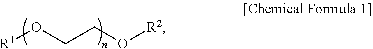

In Chemical Formula 1, R1 and R2 are independently H or an alkyl group having 1 to 5 carbon atoms, and n is an integer from 1 to 4.

In Chemical Formula 1, R1 and R2 are an alkyl group having 1 to 5 carbon atoms, preferably an alkyl group having 1 to 4 carbon atoms. In this case, the effect of reducing process by-products and the effect of improving thin film density may be increased, and step coverage and the electrical properties, insulating properties, and dielectric properties of the thin film may be excellent.

In Chemical Formula 1, n is an integer from 1 to 4, preferably an integer from 1 to 3. In this case, the effect of reducing process by-products and the effect of improving thin film density may be increased, and step coverage and the electrical properties, insulating properties, and dielectric properties of the thin film may be excellent.

For example, the step coverage improver may have a refractive index (measured at 20 to 25° C.) of 1.30 or more, 1.3 to 1.5, 1.35 to 1.48, or 1.36 to 1.46.

In this case, by forming, as a shielded area that does not remain in the thin film, a deposition layer of uniform thickness due to the difference in adsorption distribution of the shielding agent having the aforementioned structure on the substrate, the deposition rate of the thin film may be reduced, and the thin film growth rate may be appropriately reduced. Thus, even when a thin film is formed on a substrate having a complex structure under high-temperature conditions, step coverage and the thickness uniformity of a thin film may be greatly improved. In addition to a thin film precursor, the surface of the substrate may be effectively protected by preventing the adsorption of process by-products, and process by-products may be effectively removed.

In particular, as a relatively coarse thin film is formed, the growth rate of a thin film formed at the same time is greatly reduced, so that even when applied to a substrate having a complex structure, the uniformity of the thin film may be secured, and the step coverage may be greatly improved. In particular, deposition in a thin thickness is possible, and the remaining amounts of O, Si, metals, and metal oxides remaining as process by-products may be improved. In addition, even the remaining amount of carbon, which was difficult to reduce in the past, may be improved.

The step coverage improver may include one or more selected from compounds represented by Chemical Formulas 1-1 to 1-7 below. In this case, by providing a thin film-shielded area, the growth rate of a thin film may be effectively controlled, and process by-products may be effectively removed. In addition, step coverage and film quality may be improved. In addition, the uniformity of the formed thin film may be significantly improved.

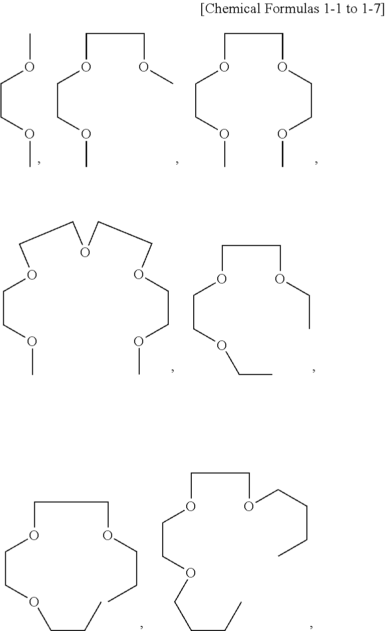

The diffusion improver may have a boiling point of 5 to 200° C., as a specific example, 50 to 150° C., preferably 100 to 140° C. In this case, the diffusion of the above-described step coverage improver may be improved. In addition, by suppressing side reactions during thin film formation and controlling the thin film growth rate, process by-products within the thin film may be reduced, thereby reducing corrosion and deterioration. In addition, film quality including thin film crystallinity may be improved, and a stoichiometric oxidation state may be reached when a metal oxide film is formed. In addition, even when a thin film is formed on a substrate with a complex structure under high-temperature conditions, step coverage and the thickness uniformity of a thin film may be greatly improved.

For example, the diffusion improver may have a solubility (25° C.) of 200 mg/L or less, preferably 50 to 400 mg/L, more preferably 135 to 175 mg/L in water. Within this range, the reactivity toward the precursor compound may be reduced, and moisture may be easily managed.

In the present disclosure, solubility may be measured without any particular limitation by a measurement method or standard commonly used in the technical field to which the present invention pertains. For example, solubility may be measured by the HPLC method using a saturated solution.

The diffusion improver, preferably one or more selected from octane, diethyl ether, and methylene chloride may have a deposition rate reduction rate of 5% or more, as a specific example, 10% or more, as calculated by Equation 1 below. In this case, by forming a deposition layer with a uniform thickness due to the difference in adsorption distribution of the step coverage improver having the above-described structure as a shielded area that does not remain in the thin film, as a relatively coarse thin film is formed, the growth rate of a thin film formed at the same time is greatly reduced, so that even when applied to a substrate having a complex structure, the uniformity of the thin film may be secured, and the step coverage may be greatly improved. In particular, deposition in a thin thickness is possible, and the remaining amounts of O, Si, metals, and metal oxides remaining as process by-products may be improved. In addition, even the remaining amount of carbon, which was difficult to reduce in the past, may be improved.

$$Deposition rate reduction rate = [ \{ ( {DR}_{i} ) - ( D {R}_{f} ) \} / ( {DR}_{i} ) ] \times 100 \qquad [ Equation 1 ]$$

In Equation 1, deposition rate (DR, Å/cycle) is the speed at which a thin film is deposited. In the deposition of a thin film formed from a precursor and a reactant, DRᵢ (initial deposition rate) is the deposition rate of the thin film formed without adding a diffusion improver. DR_{f} (final deposition rate) is the deposition rate of the thin film formed by adding the diffusion improver during the above process. Here, the deposition rate (DR) is a value measured at room temperature and pressure using an ellipsometer for a thin film with a thickness of 3 to 30 nm, and is expressed in a unit of Å/cycle.

In Equation 1, when the thin film modification composition is used and when the thin film modification composition is not used, for each case, the thin film growth rate per cycle means the thin film deposition thickness (Å/cycle) per cycle, i.e., the deposition rate. For example, the deposition rate may be obtained as an average deposition rate calculated by measuring the final thickness of a thin film with a thickness of 3 to 30 nm under room temperature and pressure conditions using an ellipsometer, and then dividing the final thickness by the total number of cycles.

In Equation 1, “when the thin film modification composition is not used” means that a thin film is manufactured by adsorbing only a precursor compound on a substrate in a thin film deposition process. As a specific example, in the thin film forming method, the above case refers to a case where a thin film is formed by omitting a step of adsorbing a thin film modification composition and a step of purging an unadsorbed thin film modification composition.

The diffusion improver, preferably one or more selected from octane, diethyl ether, and methylene chloride may have a non-uniformity of 100% or more, as a specific example, 110% or more, as calculated by Equation 2 below. In this case, by forming a deposition layer with a uniform thickness due to the difference in adsorption distribution of the step coverage improver having the above-described structure as a shielded area that does not remain in the thin film, as a relatively coarse thin film is formed, the growth rate of a thin film formed at the same time is greatly reduced, so that even when applied to a substrate having a complex structure, the uniformity of the thin film may be secured, and the step coverage may be greatly improved. In particular, deposition in a thin thickness is possible, and the remaining amounts of O, Si, metals, and metal oxides remaining as process by-products may be improved. In addition, even the remaining amount of carbon, which was difficult to reduce in the past, may be improved.

$$Non - uniformity ( \% ) = [ \text{} \{ \text{} ( Maximum thickness - Minimum thickness ) / 2 \} \times Average thickness ] \times 100 \qquad [ Equation 2 ]$$

For example, the diffusion improver may be a compound having a refractive index of 1.30 or more, 1.30 to 1.70, 1.35 to 1.60, or 1.36 to 1.50.

In this case, by appropriately shielding the adsorption of the precursor compound on the substrate, the reaction speed may be improved. In addition, even when forming a thin film on a substrate with a complex structure, step coverage and the thickness uniformity of a thin film may be greatly improved. In addition to a thin film precursor, the surface of the substrate may be effectively protected by preventing the adsorption of process by-products, and process by-products may be effectively removed.

The thin film modification composition may include the above-described step coverage improver and the diffusion improver in a weight ratio of 50:50 to 99:1, as a specific example, 60:40 to 95:5, preferably 70:30 to 92:8, still more preferably 80:20 to 90:10. In this case, as a relatively coarse thin film is formed, the growth rate of a thin film formed at the same time is greatly reduced, so that even when applied to a substrate having a complex structure, the uniformity of the thin film may be secured, and the step coverage may be greatly improved. In particular, deposition in a thin thickness is possible, and the remaining amounts of O, Si, metals, and metal oxides remaining as process by-products may be improved. In addition, even the remaining amount of carbon, which was difficult to reduce in the past, may be improved.

For reference, the diffusion improver may include octane having a boiling point of about 125.6° C., methylene chloride having a boiling point of 39.6° C., and diethyl ether having a boiling point of about 34.6° C.

The thin film modification composition may have a deposition rate reduction rate of 5% or more as calculated by Equation 1 below.

$$Deposition rate reduction rate = [ \{ ( {DR}_{i} ) - ( D {R}_{f} ) \} / ( {DR}_{i} ) ] \times 100 \qquad [ Equation 1 ]$$

In Equation 1, deposition rate (DR, Å/cycle) is the speed at which a thin film is deposited. In the deposition of a thin film formed from a precursor and a reactant, DRᵢ (initial deposition rate) is the deposition rate of the thin film formed without adding a diffusion improver. DR_{f} (final deposition rate) is the deposition rate of the thin film formed by adding the diffusion improver during the above process. Here, the deposition rate (DR) is a value measured at room temperature and pressure using an ellipsometer for a thin film with a thickness of 3 to 30 nm, and is expressed in a unit of Å/cycle.

The shielded area for thin films does not remain on the thin film.

At this time, unless otherwise specified, non-residue refers to a case where the content of C element is less than 0.1 atom %, the content of Si element is less than 0.1 atom %, the content of N element is less than 0.1 atom %, and the content of halogen element is less than 0.1 atom % when analyzed by XPS. More preferably, in the secondary-ion mass spectrometry (SIMS) measurement method or X-ray photoelectron spectroscopy (XPS) measurement method, in which measurements are performed in the depth direction of a substrate, considering the increase/decrease rate of C, N, Si, and halogen impurities before and after using the step coverage improver under the same deposition conditions, it is desirable that the increase/decrease rate of the signal sensitivity (intensity) of each element type does not exceed 5%.

For example, the thin film may include a halogen compound in an amount of 100 ppm or less. For reference, when excess halogen remains, it is not desirable because by-products, such as NH4Cl, may be generated and may remain in the thin film when using a nitriding agent under the experimental conditions of 200 to 300° C. described later.

The thin film may be used as an etching stop film, an electrode film, a dielectric film, a gate insulating film, a block oxide film, or a charge trap, without being limited thereto.

The thin film modification composition may be preferably a compound having a purity of 99.9% or more, 99.95% or more, or 99.99% or more. For reference, when a compound having a purity of less than 99% is used, impurities may remain in a thin film or cause side reactions with precursors or reactants. Accordingly, it is desirable to use a material having a purity of 99% or more.

The thin film modification composition is preferably used in the atomic layer deposition (ALD) process. In this case, the surface of a substrate may be effectively protected and process by-products may be effectively removed without interfering with the adsorption of a precursor compound.

The thin film modification composition may have preferably a density of 0.8 to 2.5 g/cm³ or 0.8 to 1.5 g/cm³ and a vapor pressure (20° C.) of 0.1 to 300 mmHg or 1 to 300 mmHg. Within this range, a shielded area may be effectively formed, and the step coverage and the thickness uniformity and film quality of a thin film may be greatly improved.

More preferably, the thin film modification composition may have a density of 0.75 to 2.0 g/cm³ or 0.8 to 1.3 g/cm³ and a vapor pressure (20° C.) of 1 to 260 mmHg. Within this range, a shielded area may be effectively formed, and the step coverage and the thickness uniformity and film quality of a thin film may be greatly improved.

In particular, as a relatively coarse thin film is formed, the growth rate of a thin film formed at the same time is greatly reduced, so that even when applied to a substrate having a complex structure, the uniformity of the thin film may be secured, and the step coverage may be greatly improved. In particular, deposition in a thin thickness is possible, and the remaining amounts of O, Si, metals, and metal oxides remaining as process by-products may be improved. In addition, even the remaining amount of carbon, which was difficult to reduce in the past, may be improved.

The method of forming a thin film according to the present invention includes a step of injecting the above-described thin film modification composition into a chamber to replace the ligand of a precursor compound adsorbed on the surface of a loaded substrate. In this case, by effectively replacing the ligand of the precursor adsorbed on the substrate, the reaction speed may be improved and the thin film growth rate may be appropriately reduced. In addition, even when forming a thin film on a substrate with a complex structure, step coverage and the thickness uniformity of a thin film may be greatly improved.

In the step of shielding the thin film modification composition on the substrate, the feeding time (sec) of the thin film modification composition on the surface of the substrate may be preferably 0.01 to 10 seconds, more preferably 0.02 to 8 seconds, still more preferably 0.04 to 6 seconds, still more preferably 0.05 to 5 seconds per cycle. Within this range, thin film growth rate may be reduced, and step coverage and economics may be excellent.

In the present disclosure, the feeding time of the precursor compound is based on a flow rate of 0.1 to 500 mg/cycle at a chamber volume of 15 to 20 L, more specifically is based on a flow rate of 0.8 to 200 mg/cycle at a chamber volume of 18 L.

As a preferred example, the method of forming a thin film may include step i) of vaporizing the above-described thin film modification composition to shield the surface of a substrate loaded in a chamber; step ii) of performing 1st purging inside the chamber with a purge gas; step iii) of vaporizing a precursor compound and adsorbing the precursor compound onto an area outside the shielded area; step iv) of performing 2nd purging inside the chamber with a purge gas; step v) of supplying a reaction gas inside the chamber; and step vi) of performing 3rd purging inside the chamber with a purge gas.

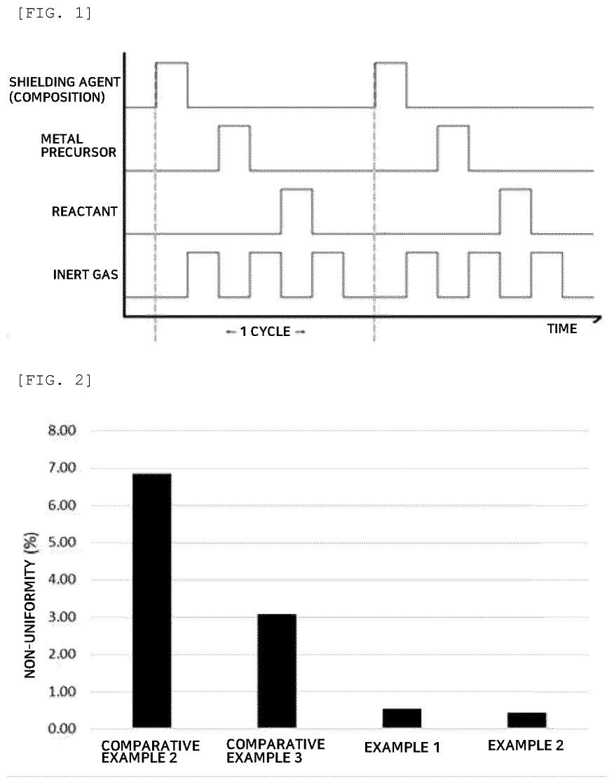

At this time, steps i) to vi) may be repeated as a unit cycle until a thin film of the desired thickness is obtained (see FIG. 1 below). In this way, in one cycle, when the thin film modification composition of the present invention is injected before the precursor compound to improve film quality, even when deposition is performed at high temperatures, the thin film growth rate may be appropriately reduced, process by-products may be effectively removed, the resistivity of the thin film may be reduced, and the step coverage may be significantly improved.

As a preferred example, in the method of forming a thin film according to the present invention, in one cycle, by introducing the thin film modification composition of the present invention before the precursor compound, the surface of the substrate may be activated, and then the precursor compound may be introduced to adsorb the precursor compound onto the substrate. In this case, even when depositing a thin film at high temperatures, process by-products may be significantly reduced and step coverage may be significantly improved by appropriately reducing the thin film growth rate. In addition, the crystallinity of the thin film may be increased, thereby reducing the resistivity of the thin film. In addition, even when applied to semiconductor devices with a large aspect ratio, the thickness uniformity of the thin film may be greatly improved, thereby ensuring the reliability of the semiconductor device.

For example, in the method of forming a thin film, when the thin film modification composition is deposited before or after the deposition of the precursor compound, depending on the needs, the unit cycle may be repeated 1 to 99,999 times, preferably 10 to 10,000 times, more preferably 50 to 5,000 times, still more preferably 100 to 2,000 times. Within this range, the desired thickness of the thin film may be obtained, and the effects intended for the present invention may be sufficiently achieved.

The precursor compound may be a molecule including one or more selected from the group consisting of Al, Si, Ti, V, Co, Ni, Cu, Zn, Ga, Ge, Se, Zr, Nb, Mo, Ru, Rh, In, Sn, Sb, Te, Hf, Ta, W, Re, Os, Ir, La, Ce, and Nd as central metal atoms (M) and one or more ligands selected from C, N, O, H, X (halogen), and Cp (cyclopentadiene). For a precursor having a vapor pressure of 1 mTorr to 100 Torr at 25° C., despite natural oxidation, the effect of forming a shielded area by the above-described oxide film reaction surface control agent may be maximized.

In the present invention, for example, the chamber may be an ALD chamber, a CVD chamber, a PEALD chamber, or a PECVD chamber.

The thin film may be a silicon oxide film, a titanium oxide film, a hafnium oxide film, a zirconium oxide film, a tungsten oxide film, an aluminum oxide film, a niobium oxide film, or a tellurium oxide film.

In the present invention, the precursor compound or precursor compound may be vaporized, injected, and then subjected to plasma post-treatment. In this case, the growth rate of thin film may be improved and process by-products may be reduced.

When the precursor compound is adsorbed while injecting the thin film modification composition onto the substrate, the amount of purge gas injected into the chamber in the step of purging the unadsorbed thin film modification composition is not particularly limited as long as the amount is an amount sufficient to remove the unadsorbed thin film modification composition. For example, the amount of purge gas may be 10 to 100,000 times, preferably 50 to 50,000 times, more preferably 100 to 10,000 times. Within this range, by sufficiently removing the unadsorbed oxide film reaction surface control agent, a thin film may be formed evenly and deterioration of film quality may be prevented. Here, the input amounts of the purge gas and the thin film modification composition are each based on one cycle, and the volume of the thin film modification composition means a volume of the vaporized thin film modification composition.

As a specific example, in the step of injecting the thin film modification composition at a flow rate of 100 mL/min (injection time 2 sec/cycle) and purging the unadsorbed thin film modification composition, a purge gas (Ar) is injected at a flow rate of 4000 mL/min (injection time 10 sec/cycle), and the injection amount of the purge gas is 40 times the injection amount of the thin film modification composition.

In the step of purging the unadsorbed precursor compound, the amount of purge gas injected into the chamber is not particularly limited as long as the amount is sufficient to remove the unadsorbed precursor compound. For example, the amount may be 10 to 10,000 times, preferably 50 to 50,000 times, more preferably 100 to 10,000 times the volume of the precursor compound injected into the chamber. Within this range, by sufficiently removing the unadsorbed precursor compound, a thin film may be formed evenly and deterioration of film quality may be prevented. Here, the input amounts of the purge gas and precursor compound are each based on one cycle, and the volume of the precursor compound refers to the volume of the vaporized precursor compound vapor.

In addition, in the purging step performed immediately after the reaction gas supply step, the amount of purge gas introduced into the chamber may be, for example, 10 to 10,000 times, preferably 50 to 50,000 times, more preferably 100 to 10,000 times the volume of reaction gas introduced into the chamber. Within this range, the desired effects may be sufficiently achieved. Here, the input amounts of purge gas and reaction gas are based on one cycle.

The thin film modification composition and the precursor compound may be transported into the chamber preferably by a VFC, DLI, or LDS method, more preferably an LDS method.

For example, the substrate loaded into the chamber may be heated to 100 to 650° C., as a specific example, 150 to 550° C. The thin film modification composition or the precursor compound may be injected onto the substrate in an unheated or heated state, or may be injected unheated and then heated during the deposition process, depending on the deposition efficiency. For example, the thin film modification composition or the precursor compound may be injected onto the substrate at 100 to 650° C. for 1 to 20 seconds.

The ratio of amount (mg/cycle) of the thin film modification composition and the precursor compound fed into the chamber may be preferably 1:1.5 to 1:20, more preferably 1:2 to 1:15, still more preferably 1:2 to 1:12, still more preferably 1:2.5 to 1:10. Within this range, step coverage may be improved, and process by-products may be greatly reduced.

The precursor material may be used in combination with a non-polar solvent.

The non-polar solvent may be an alkane or cycloalkane with 1 to 5 carbon atoms. Specifically, based on a total weight of the precursor compound and the non-polar solvent, the non-polar solvent may be included in an amount of 5 to 95% by weight, more preferably 10 to 90% by weight, still more preferably 40 to 90% by weight, most preferably 70 to 90% by weight.

Even when the above-described type of non-polar solvent is used, when the amount used exceeds the upper limit, impurities may be generated, which may increase the resistance and impurities within a thin film. When the content of the organic solvent is less than the range, the effect of improving step coverage due to solvent addition and the effect of reducing impurities such as chloride (Cl) ions may be reduced.

For example, in the method of forming a thin film, when the thin film modification composition is used, the deposition rate reduction rate expressed by Equation 1 below may be 5% or more, as a specific example, 10% or more. In this case, by forming a deposition layer with a uniform thickness due to the difference in adsorption distribution of the step coverage improver having the above-described structure as a substitution area that does not remain in the thin film, as a relatively coarse thin film is formed, the growth rate of a thin film formed at the same time is greatly reduced, so that even when applied to a substrate having a complex structure, the uniformity of the thin film may be secured, and the step coverage may be greatly improved. In particular, deposition in a thin thickness is possible, and the remaining amounts of O, Si, metals, and metal oxides remaining as process by-products may be improved. In addition, even the remaining amount of carbon, which was difficult to reduce in the past, may be improved.

$$Deposition rate reduction rate = [ \{ ( {DR}_{i} ) - ( D {R}_{f} ) \} / ( {DR}_{i} ) ] \times 100 \qquad [ Equation 1 ]$$

In Equation 1, deposition rate (DR, Å/cycle) is the speed at which a thin film is deposited. In the deposition of a thin film formed from a precursor and a reactant, DRᵢ (initial deposition rate) is the deposition rate of the thin film formed without adding a diffusion improver. DR_{f} (final deposition rate) is the deposition rate of the thin film formed by adding the diffusion improver during the above process. Here, the deposition rate (DR) is a value measured at room temperature and pressure using an ellipsometer for a thin film with a thickness of 3 to 30 nm, and is expressed in a unit of Å/cycle.

For example, in the method of forming a thin film, when the thin film modification composition is used, the non-uniformity as calculated by Equation 2 below may be 1% or less, as a specific example, 0.01 to 1%, preferably 0.1 to 0.7%. In this case, by forming a deposition layer with a uniform thickness due to the difference in adsorption distribution of the step coverage improver having the above-described structure as a substitution area that does not remain in the thin film, as a relatively coarse thin film is formed, the growth rate of a thin film formed at the same time is greatly reduced, so that even when applied to a substrate having a complex structure, the uniformity of the thin film may be secured, and the step coverage may be greatly improved. In particular, deposition in a thin thickness is possible, and the remaining amounts of O, Si, metals, and metal oxides remaining as process by-products may be improved. In addition, even the remaining amount of carbon, which was difficult to reduce in the past, may be improved.

$$Non - uniformity ( \% ) = [ \text{} \{ \text{} ( Maximum thickness - Minimum thickness ) / 2 \} \times Average thickness ] \times 100 \qquad [ Equation 2 ]$$

The maximum and minimum thicknesses were selected by measuring a thin film using an ellipsometer used for the above-described deposition rate reduction rate.

For example, it may be selected by measuring the thicknesses of 10 randomly selected locations, and preferably, it may be selected by measuring the thicknesses of 4 locations on the east, west, south, and north edges of a 300 mm wafer and 1 location in the center.

In the method of forming a thin film, the residual halogen intensity (c/s) in the thin film may be preferably 100,000 or less, more preferably 70,000 or less, still more preferably 50,000 or less, still more preferably 10,000 or less, as a preferred example, 5,000 or less, still more preferably 1,000 to 4,000, still more preferably 1,000 to 3,800 as measured using a thin film having a thickness of 100 Å according to SIMS. Within this range, corrosion and deterioration may be effectively prevented.

In the present disclosure, purging may be performed at preferably 1,000 to 50,000 sccm (standard cubic centimeter per minute), more preferably 2,000 to 30,000 sccm, still more preferably 2,500 to 15,000 sccm. Within this range, the thin film growth rate per cycle may be appropriately controlled. In addition, since deposition is performed as an atomic mono-layer or nearly an atomic mono-layer, the film quality may be improved.

The atomic layer deposition (ALD) process is very advantageous in manufacturing integrated circuits (ICs) that require a high aspect ratio. In particular, the ALD process has advantages such as excellent conformality, uniformity, and precise thickness control due to the self-limiting thin film growth mechanism.

For example, the method of forming a thin film may be performed at a deposition temperature of 50 to 800° C., preferably 300 to 700° C., more preferably 400 to 650° C., still more preferably 400 to 600° C. Within this range, a thin film with excellent film quality may be grown while implementing ALD process characteristics.

For example, the method of forming a thin film may be performed at a deposition pressure of 0.01 to 20 Torr, preferably 0.1 to 20 Torr, more preferably 0.1 to 10 Torr, most preferably 0.3 to 7 Torr. Within this range, a thin film having a uniform thickness may be obtained.

In the present disclosure, the deposition temperature and the deposition pressure may be measured as temperature and pressure formed within the deposition chamber, or as temperature and pressure applied to the substrate within the deposition chamber.

The method of forming a thin film may preferably include a step of increasing the temperature inside the chamber to the deposition temperature before introducing the thin film modification composition into the chamber; and/or a step of purging by injecting an inert gas into the chamber before introducing the thin film modification composition into the chamber.

In addition, as a thin film manufacturing device capable of implementing the thin film manufacturing method, the present invention may include a thin film manufacturing device including an ALD chamber, a first vaporizer for vaporizing the thin film modification composition, a first transport means for transferring the vaporized thin film modification composition into the ALD chamber, a second vaporizer for vaporizing the thin film precursor, and a second transport means for transferring the vaporized thin film precursor into the ALD chamber. Here, vaporizers and transport means commonly used in the technical field to which the present invention pertains may be used in the present invention without particular limitation.

When necessary, the first vaporizer of vaporizing a thin film modification composition may be composed of at least two types: a vaporizer for vaporizing a step coverage improver and a vaporizer for vaporizing a diffusion improver.

As a specific example, the method of forming a thin film is explained. First, a substrate on which a thin film is to be formed is placed in a deposition chamber capable of atomic layer deposition.

The substrate may include a semiconductor substrate, such as a silicon substrate or a silicon oxide substrate.

The substrate may further have a conductive layer or an insulating layer formed on the upper portion thereof.

To deposit a thin film on the substrate positioned in the deposition chamber, the above-described thin film modification composition, a precursor compound, or a mixture of the precursor compound and a non-polar solvent is prepared.

The prepared thin film modification composition (for example, step coverage improver) is injected into a vaporizer, is transformed into a vapor phase, is transferred into a deposition chamber, and is adsorbed on a substrate. Then, purging is performed to remove the unadsorbed thin film modification composition.

Next, the prepared precursor compound or mixture of the precursor compound and a non-polar solvent (composition for forming a thin film) is injected into the vaporizer, is transformed into a vapor phase, is transferred into the deposition chamber, and is adsorbed on the substrate. Then, shielding is performed by the preinjected step coverage improver, and the unadsorbed precursor compound or mixture of the precursor compound and a non-polar solvent is purged.

In the present disclosure, for example, the thin film modification composition and the precursor compound (composition for forming a thin film) may be delivered into the deposition chamber by vapor flow control (VFC) using mass flow control (MFC) or a liquid delivery system (LDS) using liquid mass flow control (LMFC), preferably an LDS method.

At this time, a mixed gas of one or more selected from the group consisting of argon (Ar), nitrogen (N₂), and helium (He) may be used as a carrier gas or dilution gas to move the thin film modification composition and the precursor compound onto the substrate, without being limited thereto.

In the present disclosure, for example, an inert gas, preferably the carrier gas or dilution gas may be used as the purge gas.

Next, a reaction gas is supplied. A reaction gas commonly used in the technical field to which the present invention pertains may be used in the present invention without particular limitation. Preferably, the reaction gas may contain a nitriding agent. The nitriding agent and the precursor compound adsorbed on the substrate react to form a nitride film.

Preferably, the nitriding agent may be nitrogen gas (N₂), hydrazine gas (N₂H₄), or a mixture of nitrogen gas and hydrogen gas.

Next, unreacted residual reaction gas is purged using an inert gas. Accordingly, in addition to excess reaction gas, generated byproducts may also be removed.

As described above, in the method of forming a thin film, for example, a step of supplying a step coverage improver onto a substrate; a step of purging the unadsorbed step coverage improver; a step of adsorbing a precursor compound/composition for forming a thin film on the substrate; a step of purging the unadsorbed precursor compound; a step of adsorbing a diffusion improver onto the substrate; a step of purging the unadsorbed diffusion improver; a step of supplying a reaction gas; and a step of purging the residual reaction gas may be set as a unit cycle. To form a thin film of desired thickness, the unit cycle may be repeated.

For example, the unit cycle may be repeated 1 to 99,999 times, preferably 10 to 1,000 times, more preferably 50 to 5,000 times, still more preferably 100 to 2,000 times. Within this range, the desired thin film properties may be well expressed.

In addition, the present invention provides a semiconductor substrate, and the semiconductor substrate is fabricated by the thin film formation method. In this case, the step coverage and thickness uniformity of a thin film may be excellent, and density and electrical properties may be excellent.

For example, the manufactured thin film may have a thickness of preferably 0.1 to 20 nm, preferably 0.5 to 20 nm, more preferably 1.5 to 15 nm, still more preferably 2 to 10 nm. Within this range, thin film properties may be excellent.

The thin film may have a carbon impurity content of preferably 5,000 counts/sec or less or 1 to 3,000 counts/sec, still more preferably 10 to 1,000 counts/sec, still more preferably 50 to 500 counts/sec. Within this range, thin film properties may be excellent, and thin film growth rate may be reduced.

For example, the thin film may have a step coverage of 90% or more, preferably 92% or more, more preferably 95% or more. Within this range, since even a thin film of complex structure may be easily deposited on a substrate, the thin film may be applied to next-generation semiconductor devices.

The manufactured thin film may have a thickness of preferably 20 nm or less, a dielectric constant of 5 to 29 based on a thin film thickness of 10 nm, a carbon, nitrogen, and halogen content of 5,000 counts/sec or less, and a step coverage of 90% or more. Within this range, the thin film may have excellent performance as a dielectric film or blocking film, without being limited thereto.

For example, when necessary, the thin film may have a multilayer structure of two or more layers, preferably a multilayer structure of two or three layers. As a specific example, the multilayer film with a two-layer structure may have a lower layer-middle layer structure, and the multilayer film with a three-layer structure may have a lower layer-middle layer-upper layer structure.

For example, the lower layer may be formed of one or more selected from the group consisting of Si, SiO₂, MgO, Al₂O₃, CaO, ZrSiO₄, ZrO₂, HfSiO₄, Y₂O₃, HfO₂, LaLuO₂, Si₃N₄, SrO, La₂O₃, Ta₂O₅, BaO, and TiO₂.

For example, the middle layer may be formed of TiₓN_{y}, preferably TN.

For example, the upper layer may be formed of one or more selected from the group consisting of W and Mo.

Hereinafter, preferred examples and drawings are presented to help understand the present invention, but the following examples and drawings are only illustrative of the present invention, and it is obvious to those skilled in the art that various changes and modifications are possible within the scope and technical idea of the present invention. Such changes and modifications fall within the scope of the appended patent claims.

### EXAMPLES

### Examples 1 and 2, Comparative Examples 1 to 3

An ALD deposition process was performed according to FIG. 1 below using the components shown in Table 1 below.

FIG. 1 below is a schematic diagram showing a deposition process sequence according to the present invention, focusing on one cycle.

Specifically, as the step coverage improver, a compound represented by Chemical Formula 1-1 below and a compound represented by Chemical Formula 1-2 below were prepared. As the diffusion improver, a compound represented by Chemical Formula 4-1 below, a compound represented by Chemical Formula 4-2 below, and a compound represented by Chemical Formula 4-3 below were prepared.

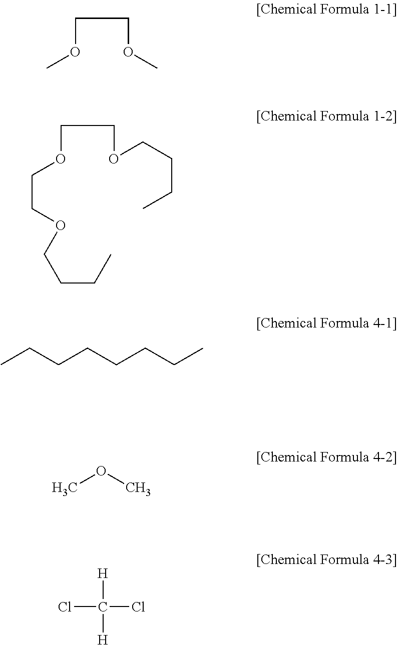

The thin film modification composition was prepared by mixing 90 parts by weight of the compound represented by Chemical Formula 1-1 and 10 parts by weight of the compound represented by Chemical Formula 4-1, and is designated as Composition 1 in Table 1.

The thin film modification composition was prepared by mixing 90 parts by weight of the compound represented by Chemical Formula 1-2 and 10 parts by weight of the compound represented by Chemical Formula 4-1, and is designated as Composition 2 in Table 2.

In addition, as the precursor, trimethylaluminum (indicated as TMA in the table below) was prepared.

As shown in FIG. 1 below, argon was introduced into the chamber at a rate of 5000 ml/min, and a vacuum pump was used to create a thin, inert atmosphere by maintaining the pressure inside the chamber at 2.5 Torr.

The thin film modification composition shown in Table 1 below was placed in a canister and the partial pressure and temperature were adjusted to achieve the injection amount (mg/cycle). Then, the thin film modification composition was applied into a substrate loaded into the chamber for 1 second, and the chamber was purged for 10 seconds.

Next, the precursor compound was placed in the canister and injected into the deposition chamber through a vapor flow controller (VFC) as shown in Table 1, and the chamber was purged for 10 seconds.

Next, the concentration of O₃ in O₂ as a reactive gas was adjusted to 200 g/m³ and the reactive gas was introduced into the deposition chamber as shown in Table 1, and the chamber was purged for 10 seconds. At this time, the substrate on which a thin film was to be formed was heated under the temperature conditions shown in Table 1 below.

This process was repeated 100 to 400 times to form a self-limiting atomic layer thin film with a thickness of 10 nm.

For thin films obtained in Examples 1 and 2 and Comparative Examples 1 to 3, deposition rate reduction rate (D/R reduction rate), SIMS C impurities, and step coverage were measured according to the following methods.

*Deposition rate reduction rate (D/R (dep. rate) reduction rate): The deposition rate reduction rate represents the rate of reduction in the deposition rate after the shielding agent is added compared to the deposition rate before the thin film modification composition is added, and was calculated as a percentage using the measured A/cycle values.

Specifically, using an ellipsometer which is a device capable of measuring optical properties such as thickness and refractive index of the thin film by using the polarization characteristics of light for the manufactured thin film, the thickness of the thin film was measured. Then, the thickness of the thin film deposited per cycle was calculated by dividing the measured thickness by the number of cycles. Based on these results, the thin film growth rate reduction rate was calculated. Specifically, the calculation was performed using Equation 1 below.

$$Deposition rate reduction rate = [ \{ ( {DR}_{i} ) - ( D {R}_{f} ) \} / ( {DR}_{i} ) ] \times 100 \qquad [ Equation 1 ]$$

In Equation 1, deposition rate (DR, Å/cycle) is the speed at which a thin film is deposited. In the deposition of a thin film formed from a precursor and a reactant, DRᵢ (initial deposition rate) is the deposition rate of the thin film formed without adding a diffusion improver. DR_{f} (final deposition rate) is the deposition rate of the thin film formed by adding the diffusion improver during the above process. Here, the deposition rate (DR) is a value measured at room temperature and pressure using an ellipsometer for a thin film with a thickness of 3 to 30 nm, and is expressed in a unit of Å/cycle.

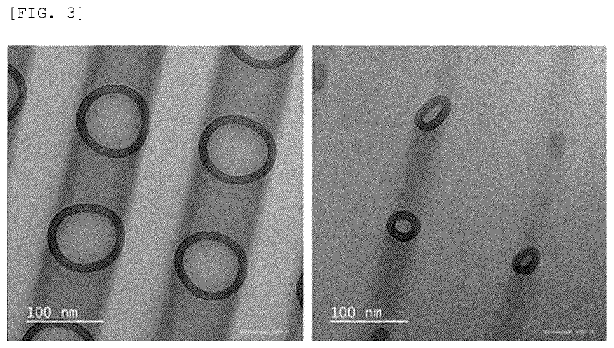

*To evaluate the non-uniformity, the maximum and minimum thicknesses were selected from the thicknesses of the thin film measured by the ellipsometer, and the results calculated using Equation 2 below are shown in Table 1 below and FIG. 2. Specifically, the thickness of four locations on the east, west, south, and north edges of a 300 mm wafer and the thickness of one location in the center were measured.

$$Non - uniformity ( \% ) = [ \text{} \{ \text{} ( Maximum thickness - Minimum thickness ) / 2 \} \times Average thickness ] \times 100 \qquad [ Equation 2 ]$$

*SIMS (Secondary-ion mass spectrometry) C impurities: The C impurity value was checked from the SIMS graph by considering the C impurity content (counts) when the thin film was axially penetrated by an ion sputter and the sputter time was 50 seconds with little contamination on the substrate surface layer.

*Step coverage (%) TEM measurements were taken on specimens cut horizontally at positions 100 nm downward from the upper portion (left drawing) and 100 nm upward from the lower portion (right drawing) of thin films deposited on a substrate of a complex structure with an aspect ratio of 22:1 by Examples 1 and 2 and Comparative Examples 1 to 3, and the step coverage was measured by Equation 3 below.

$$Step coverage \% = ( Thickness deposited on inner wall of lower portion \text{} / Thickness deposited on inner wall of upper portion ) \times 100 \qquad [ Equation 3 ]$$

Specifically, on a substrate of a complex structure with an upper portion diameter of 90 nm, a lower portion diameter of 65 nm, a via hole depth of approximately 2000 nm, an aspect ratio of 22:1, a deposition process was performed using diffusion improving agent application conditions. Then, to confirm the thickness uniformity and step coverage deposited inside the vertically formed via hole, a specimen was prepared by cutting horizontally at a position 100 nm downward from the upper portion and at a position 100 nm upward from the lower portion. Then, the specimen was observed using a transmission electron microscope (TEM), and the results are shown in Table 1 and FIG. 3 below.

TABLE 1

| | | | | | | | Step | | | | |
|---|---|---|---|---|---|---|---|---|---|---|---|
| | | | Thin | | | Reaction | coverage | | | | |
| | | | film | | Precursor | gas | improver | Deposition | Non- | | Step |
| | | Reaction | modification | Deposition | injection | injection | injection | rate | uniformity | Equation 1 | coverage |
| No. | Precursor | gas | composition | temperature | condition | condition | condition | (Å/cycle) | (%) | calculation | (S/C, %) |
| Compar- | TMA | O₃ | — | 400° C. | VFC, 3 s, | 5 s, | — | 0.75 | 1.20 | — | 95.0 |
| ative | | | | | 100 sccm | 500 sccm | | | | | |
| Example1 | | | | | | | | | | | |
| Compar- | TMA | O₃ | Chemical | 400° C. | VFC, 3 s, | 5 s, | 10 mg/cycle | 0.66 | 6.84 | 12% | N/A |
| ative | | | Formula 1-1 | | 100 sccm | 500 sccm | | | | | |
| Example2 | | | | | | | | | | | |
| Compar- | TMA | O₃ | Chemical | 400° C. | VFC, 3 s, | 5 s, | 10 mg/cycle | 0.55 | 3.08 | 26% | N/A |
| ative | | | Formula 1-2 | | 100 sccm | 500 sccm | | | | | |
| Example3 | | | | | | | | | | | |
| Example1 | TMA | O₃ | Compo- | 420° C. | VFC, 3 s, | 5 s, | 10 mg/cycle | 0.66 | 0.53 | 12% | 100.6 |
| | | | sition 1 | | 100 sccm | 500 sccm | | | | | |
| Example2 | TMA | O₃ | Compo- | 420° C. | VFC, 3 s, | 5 s, | 10 mg/cycle | 0.54 | 0.42 | 27% | N/A |
| | | | sition 2 | | 100 sccm | 500 sccm | | | | | |

As shown in Table 1, Examples 1 and 2 using the thin film modification composition according to the present invention exhibited deposition rate reduction rate equivalent to or similar to that of Comparative Examples 1 to 3 in which the thin film modification composition according to the present invention was not used. In addition, compared to Comparative Examples 1 to 3, in Examples 1 and 2, non-uniformity was significantly improved. In particular, compared to Comparative Example 1 in which the thin film modification composition of the present invention was not used or Comparative Examples 2 and 3 in which the step coverage improver was used alone, Examples 1 and 2 using the thin film modification composition of the present invention exhibited excellent uniformity (100% or more) (see FIG. 2 below).

In addition, as shown in FIG. 3, Example 1 using the thin film modification composition of the present invention exhibited a step coverage of 100.6%, showing significantly improved step coverage, compared to Comparative Example 1 (95.0%) without using the thin film modification composition of the present invention.

1. A thin film modification composition, comprising 50 to 99 parts by weight of a step coverage improver comprising a compound represented by Chemical Formula 1 below and 1 to 50 parts by weight of a diffusion improver. 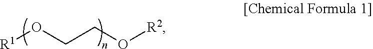
wherein R1 and R2 are independently H or an alkyl group having 1 to 5 carbon atoms, and n is an integer from 1 to 4.

2. The thin film modification composition according to claim 1, wherein the step coverage improver comprises one or more selected from compounds represented by Chemical Formulas 1-1 to 1-7 below.

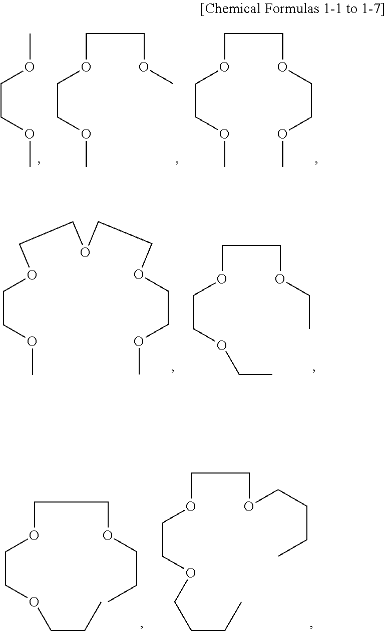

3. The thin film modification composition according to claim 1, wherein the diffusion improver has a boiling point of 5 to 200° C.

4. The thin film modification composition according to claim 1, wherein the diffusion improver comprises one or more selected from octane, diethyl ether, methylene chloride, toluene, hexane, and ethanol.

5. The thin film modification composition according to claim 1, wherein the thin film controls a reaction surface of a thin film formed from one or more precursor compounds selected from the group consisting of Al, Si, Ti, V, Co, Ni, Cu, Zn, Ga, Ge, Se, Zr, Nb, Mo, Ru, Rh, In, Sn, Sb, Te, Hf, Ta, W, Re, Os, Ir, La, Ce, and Nd.

6. A method of forming a thin film, comprising injecting a thin film modification composition comprising 50 to 99 parts by weight of a step coverage improver comprising a compound represented by Chemical Formula 1 below and 1 to 50 parts by weight of a diffusion improver into a chamber to shield a surface of a loaded substrate. 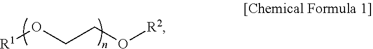
wherein R1 and R2 are independently H or an alkyl group having 1 to 5 carbon atoms, and n is an integer from 1 to 4.

7. The method according to claim 6, wherein the chamber is an ALD chamber, a CVD chamber, a PEALD chamber, or a PECVD chamber.

8. The method according to claim 6, wherein the thin film modification composition is transferred into the chamber by a VFC, DLI, or LDS method, and the thin film is a silicon nitride film, a silicon oxide film, a titanium nitride film, a titanium oxide film, a tungsten nitride film, a molybdenum nitride film, a hafnium oxide film, a zirconium oxide film, a tungsten oxide film, or an aluminum oxide film.

9. A semiconductor substrate, comprising the thin film manufactured using the method according to claim 6.

10. The semiconductor substrate according to claim 9, wherein the thin film has a multilayer structure of two or more layers.

11. A semiconductor device, comprising the semiconductor substrate according to claim 9.
